SEQUENCE LISTING (146 sequences)

SEQ ID NO: 1 - DNA, 19 bp, synthetic construct
  source 1..19: mol_type = other DNA
aaggccccttcctgtgact

SEQ ID NO: 2 - DNA, 19 bp, synthetic construct
  source 1..19: mol_type = other DNA
ttgctcagggccacttcca

SEQ ID NO: 3 - DNA, 19 bp, synthetic construct
  source 1..19: mol_type = other DNA
gcagtgagccaggatggtg

SEQ ID NO: 4 - DNA, 22 bp, synthetic construct
  source 1..22: mol_type = other DNA
gcaggaattgcacaattgcaca

SEQ ID NO: 5 - DNA, 22 bp, synthetic construct
  source 1..22: mol_type = other DNA
cacctgcagtcttaagacacct

SEQ ID NO: 6 - DNA, 22 bp, synthetic construct
  source 1..22: mol_type = other DNA
ctgatggcaggtaaggtgagtt

SEQ ID NO: 7 - DNA, 22 bp, synthetic construct
  source 1..22: mol_type = other DNA
tctcttctccagactgcaagga

SEQ ID NO: 8 - DNA, 22 bp, synthetic construct
  source 1..22: mol_type = other DNA
tgtggaagctttggatccactc

SEQ ID NO: 9 - DNA, 24 bp, synthetic construct
  source 1..24: mol_type = other DNA
tgttccattgatctatgtgcctgt

SEQ ID NO: 10 - DNA, 21 bp, synthetic construct
  source 1..21: mol_type = other DNA
cctgaatagccaaggccatcc

SEQ ID NO: 11 - DNA, 21 bp, synthetic construct
  source 1..21: mol_type = other DNA
caggctggtgaaacctgatag

SEQ ID NO: 12 - DNA, 22 bp, synthetic construct
  source 1..22: mol_type = other DNA
ggttagaccctagtctctcgtt

SEQ ID NO: 13 - DNA, 22 bp, synthetic construct
  source 1..22: mol_type = other DNA
acagtctctgtgacactgacca

SEQ ID NO: 14 - DNA, 22 bp, synthetic construct
  source 1..22: mol_type = other DNA
tgctctctgctgatttgacagg

SEQ ID NO: 15 - DNA, 23 bp, synthetic construct
  source 1..23: mol_type = other DNA
ccatccccgacctagattttcag

SEQ ID NO: 16 - DNA, 23 bp, synthetic construct
  source 1..23: mol_type = other DNA
ctgtactctggcctatgtgacag

SEQ ID NO: 17 - DNA, 23 bp, synthetic construct
  source 1..23: mol_type = other DNA
acaagaaggcaaggtagacgaat

SEQ ID NO: 18 - DNA, 24 bp, synthetic construct
  source 1..24: mol_type = other DNA
tggtccagaaggtctaatcaatca

SEQ ID NO: 19 - DNA, 25 bp, synthetic construct
  source 1..25: mol_type = other DNA
ctggaacaaaataggtggtgaggta

SEQ ID NO: 20 - DNA, 25 bp, synthetic construct
  source 1..25: mol_type = other DNA
ccaaagacagatattgcatctccca

SEQ ID NO: 21 - DNA, 24 bp, synthetic construct
  source 1..24: mol_type = other DNA
agaagtccttgctattgctctgag

SEQ ID NO: 22 - DNA, 22 bp, synthetic construct
  source 1..22: mol_type = other DNA
tccagcttggacaagagcagaa

SEQ ID NO: 23 - DNA, 22 bp, synthetic construct
  source 1..22: mol_type = other DNA
acccacacttgtttacccaagg

SEQ ID NO: 24 - DNA, 22 bp, synthetic construct
  source 1..22: mol_type = other DNA
gactgtctgatgccaaagccta

SEQ ID NO: 25 - DNA, 25 bp, synthetic construct
  source 1..25: mol_type = other DNA
gctcaataattctacaggggtctgt

SEQ ID NO: 26 - DNA, 26 bp, synthetic construct
  source 1..26: mol_type = other DNA
gcttttctctgttttcccattttcac

SEQ ID NO: 27 - DNA, 29 bp, synthetic construct
  source 1..29: mol_type = other DNA
tgctcctgggaagttataaaggtatttaa

SEQ ID NO: 28 - DNA, 33 bp, synthetic construct
  source 1..33: mol_type = other DNA
tgaggcattttataccttagcatggatttatat

SEQ ID NO: 29 - DNA, 23 bp, synthetic construct
  source 1..23: mol_type = other DNA
tgtgactagtggctactgtacca

SEQ ID NO: 30 - DNA, 23 bp, synthetic construct
  source 1..23: mol_type = other DNA
gaaggcaaaggtcagacctcttt

SEQ ID NO: 31 - DNA, 20 bp, synthetic construct
  source 1..20: mol_type = other DNA
cgagcacacacagacacaca

SEQ ID NO: 32 - DNA, 21 bp, synthetic construct
  source 1..21: mol_type = other DNA
ctctgtctctctctggctcgt

SEQ ID NO: 33 - DNA, 22 bp, synthetic construct
  source 1..22: mol_type = other DNA
tctaggattttccccaccctct

SEQ ID NO: 34 - DNA, 22 bp, synthetic construct
  source 1..22: mol_type = other DNA
tgagggacatgcatgattctcc

SEQ ID NO: 35 - DNA, 23 bp, synthetic construct
  source 1..23: mol_type = other DNA
aacactccacagtctagcctcag

SEQ ID NO: 36 - DNA, 24 bp, synthetic construct
  source 1..24: mol_type = other DNA
gacagaaggcattccattgagagt

SEQ ID NO: 37 - DNA, 25 bp, synthetic construct
  source 1..25: mol_type = other DNA
gctaagggaactcatttccatcaga

SEQ ID NO: 38 - DNA, 22 bp, synthetic construct
  source 1..22: mol_type = other DNA
caagcctccaaaatgaggctct

SEQ ID NO: 39 - DNA, 24 bp, synthetic construct
  source 1..24: mol_type = other DNA
ttacacaggttggagcatcttgga

SEQ ID NO: 40 - DNA, 24 bp, synthetic construct
  source 1..24: mol_type = other DNA
atgaggctttgtgaggtgtgattc

SEQ ID NO: 41 - DNA, 23 bp, synthetic construct
  source 1..23: mol_type = other DNA
agccttcttcaacgtctgtatct

SEQ ID NO: 42 - DNA, 24 bp, synthetic construct
  source 1..24: mol_type = other DNA
ctttgagtgccaacatctatcttc

SEQ ID NO: 43 - DNA, 21 bp, synthetic construct
  source 1..21: mol_type = other DNA
tcagtggcatatccagggtca

SEQ ID NO: 44 - DNA, 21 bp, synthetic construct
  source 1..21: mol_type = other DNA
atggtgctggaacaactggac

SEQ ID NO: 45 - DNA, 29 bp, synthetic construct
  source 1..29: mol_type = other DNA
gagagagaaagaaagaaaggaaagaaagg

SEQ ID NO: 46 - DNA, 24 bp, synthetic construct
  source 1..24: mol_type = other DNA
ctgttagctatgctggtctcaagc

SEQ ID NO: 47 - DNA, 22 bp, synthetic construct
  source 1..22: mol_type = other DNA
atgacctcttctctgcctggaa

SEQ ID NO: 48 - DNA, 22 bp, synthetic construct
  source 1..22: mol_type = other DNA
actgagttcctgcctcgaagta

SEQ ID NO: 49 - DNA, 21 bp, synthetic construct
  source 1..21: mol_type = other DNA
cacagtgctcaatgcagcttc

SEQ ID NO: 50 - DNA, 21 bp, synthetic construct
  source 1..21: mol_type = other DNA
aagctgacagcctggttactg

SEQ ID NO: 51 - DNA, 22 bp, synthetic construct
  source 1..22: mol_type = other DNA
accccttctgcctactattcca

SEQ ID NO: 52 - DNA, 22 bp, synthetic construct
  source 1..22: mol_type = other DNA
gaaggaaggaaggaaggaacga

SEQ ID NO: 53 - DNA, 22 bp, synthetic construct
  source 1..22: mol_type = other DNA
cctggaaattgacattggcaca

SEQ ID NO: 54 - DNA, 22 bp, synthetic construct
  source 1..22: mol_type = other DNA
atggctgacctaaggcctaaga

SEQ ID NO: 55 - DNA, 22 bp, synthetic construct
  source 1..22: mol_type = other DNA
tgggtagagggcagtaaagttg

SEQ ID NO: 56 - DNA, 22 bp, synthetic construct
  source 1..22: mol_type = other DNA
gaagggtgtttacgcctgtaga

SEQ ID NO: 57 - DNA, 32 bp, synthetic construct
  source 1..32: mol_type = other DNA
gaagaaaatatttgtaaaccatgtatccgatg

SEQ ID NO: 58 - DNA, 31 bp, synthetic construct
  source 1..31: mol_type = other DNA
gtctttttggatagtgatctagctaagagat

SEQ ID NO: 59 - DNA, 21 bp, synthetic construct
  source 1..21: mol_type = other DNA
cgacagagtggaccttgtctc

SEQ ID NO: 60 - DNA, 20 bp, synthetic construct
  source 1..20: mol_type = other DNA
tgctgcccaaaacagatcca

SEQ ID NO: 61 - DNA, 23 bp, synthetic construct
  source 1..23: mol_type = other DNA
gggcatctgcaaaaatcctacag

SEQ ID NO: 62 - DNA, 26 bp, synthetic construct
  source 1..26: mol_type = other DNA
acctgtgactcgctaaacttatttac

SEQ ID NO: 63 - DNA, 22 bp, synthetic construct
  source 1..22: mol_type = other DNA
aaaaccttgtgtggttggcatg

SEQ ID NO: 64 - DNA, 21 bp, synthetic construct
  source 1..21: mol_type = other DNA
ctagtgcagtggcacagttca

SEQ ID NO: 65 - DNA, 23 bp, synthetic construct
  source 1..23: mol_type = other DNA
cccttcccttcctctttctcttc

SEQ ID NO: 66 - DNA, 22 bp, synthetic construct
  source 1..22: mol_type = other DNA
gttcttttgtcagccctcacct

SEQ ID NO: 67 - DNA, 23 bp, synthetic construct
  source 1..23: mol_type = other DNA
agcgataagagggaaactgggta

SEQ ID NO: 68 - DNA, 24 bp, synthetic construct
  source 1..24: mol_type = other DNA
gccaggaatattctgtaggatgct

SEQ ID NO: 69 - DNA, 22 bp, synthetic construct
  source 1..22: mol_type = other DNA
aacacatcagtcagcaacaggt

SEQ ID NO: 70 - DNA, 22 bp, synthetic construct
  source 1..22: mol_type = other DNA
tagcaactcaggaggctgagat

SEQ ID NO: 71 - DNA, 24 bp, synthetic construct
  source 1..24: mol_type = other DNA
atcacaagatggtctggaagaaga

SEQ ID NO: 72 - DNA, 22 bp, synthetic construct
  source 1..22: mol_type = other DNA
aggttgcagtgaggtgagattg

SEQ ID NO: 73 - DNA, 22 bp, synthetic construct
  source 1..22: mol_type = other DNA
gcactcacccagatgattgctt

SEQ ID NO: 74 - DNA, 22 bp, synthetic construct
  source 1..22: mol_type = other DNA
gttccactggaaccacgtaaca

SEQ ID NO: 75 - DNA, 23 bp, synthetic construct
  source 1..23: mol_type = other DNA
gatcaggaatgcaaatgcacaca

SEQ ID NO: 76 - DNA, 22 bp, synthetic construct
  source 1..22: mol_type = other DNA
gagttgaccgacaagtcttggt

SEQ ID NO: 77 - DNA, 22 bp, synthetic construct
  source 1..22: mol_type = other DNA
gatccctgttcttgcacttgct

SEQ ID NO: 78 - DNA, 22 bp, synthetic construct
  source 1..22: mol_type = other DNA
gtgactgatgctgagttcctgg

SEQ ID NO: 79 - DNA, 22 bp, synthetic construct
  source 1..22: mol_type = other DNA
atggcctttctgaccctacctt

SEQ ID NO: 80 - DNA, 22 bp, synthetic construct
  source 1..22: mol_type = other DNA
accagctgaatttcccagtctg

SEQ ID NO: 81 - DNA, 23 bp, synthetic construct
  source 1..23: mol_type = other DNA
ctgaaactctttctcaccccctt

SEQ ID NO: 82 - DNA, 27 bp, synthetic construct
  source 1..27: mol_type = other DNA
agagtcacagtaaatgttacagaactt

SEQ ID NO: 83 - DNA, 21 bp, synthetic construct
  source 1..21: mol_type = other DNA
ggcactttgtaagctgcaacg

SEQ ID NO: 84 - DNA, 24 bp, synthetic construct
  source 1..24: mol_type = other DNA
agcacagtcatatatgtgagtgcc

SEQ ID NO: 85 - DNA, 28 bp, synthetic construct
  source 1..28: mol_type = other DNA
actaggtaggagttctaggttctaagtg

SEQ ID NO: 86 - DNA, 27 bp, synthetic construct
  source 1..27: mol_type = other DNA
ggttcaaaataagacccagcacaatag

SEQ ID NO: 87 - DNA, 23 bp, synthetic construct
  source 1..23: mol_type = other DNA
tgccccaaatgcttatctttgag

SEQ ID NO: 88 - DNA, 22 bp, synthetic construct
  source 1..22: mol_type = other DNA
cttgaacccaagaggcgtaagt

SEQ ID NO: 89 - DNA, 22 bp, synthetic construct
  source 1..22: mol_type = other DNA
atattgaccaggcctagggagt

SEQ ID NO: 90 - DNA, 21 bp, synthetic construct
  source 1..21: mol_type = other DNA
tacgcacaaacacatgtcgga

SEQ ID NO: 91 - DNA, 24 bp, synthetic construct
  source 1..24: mol_type = other DNA
atcatataacatcctgtccaagcc

SEQ ID NO: 92 - DNA, 24 bp, synthetic construct
  source 1..24: mol_type = other DNA
gacgtttctgtaaatgctgaactc

SEQ ID NO: 93 - DNA, 22 bp, synthetic construct
  source 1..22: mol_type = other DNA
agatccaacaacagcttgcact

SEQ ID NO: 94 - DNA, 23 bp, synthetic construct
  source 1..23: mol_type = other DNA
caagatgtgagttcccttggtct

SEQ ID NO: 95 - DNA, 22 bp, synthetic construct
  source 1..22: mol_type = other DNA
actccaggccaaatgaaattgc

SEQ ID NO: 96 - DNA, 24 bp, synthetic construct
  source 1..24: mol_type = other DNA
cagatcctgagatatgtggaaaga

SEQ ID NO: 97 - DNA, 24 bp, synthetic construct
  source 1..24: mol_type = other DNA
ggcaggagaatctcgctttaactc

SEQ ID NO: 98 - DNA, 25 bp, synthetic construct
  source 1..25: mol_type = other DNA
ctgagagactcaaagctttgagtgt

SEQ ID NO: 99 - DNA, 25 bp, synthetic construct
  source 1..25: mol_type = other DNA
ctcatagagttacctttcacgcaca

SEQ ID NO: 100 - DNA, 26 bp, synthetic construct
  source 1..26: mol_type = other DNA
agcagtttcacaggattaatgagtct

SEQ ID NO: 101 - DNA, 22 bp, synthetic construct
  source 1..22: mol_type = other DNA
ctattgggagaggctggctttg

SEQ ID NO: 102 - DNA, 24 bp, synthetic construct
  source 1..24: mol_type = other DNA
ccaagatgttgataggaggaagtt

SEQ ID NO: 103 - DNA, 23 bp, synthetic construct
  source 1..23: mol_type = other DNA
ccaagttgcgtctggtttaactg

SEQ ID NO: 104 - DNA, 22 bp, synthetic construct
  source 1..22: mol_type = other DNA
tccttaatgtcccactgggcta

SEQ ID NO: 105 - DNA, 24 bp, synthetic construct
  source 1..24: mol_type = other DNA
ctgttttgccttgttgagaggttg

SEQ ID NO: 106 - DNA, 26 bp, synthetic construct
  source 1..26: mol_type = other DNA
tccttgaaaactacacttgcataagg

SEQ ID NO: 107 - DNA, 22 bp, synthetic construct
  source 1..22: mol_type = other DNA
tgacaagagcaaaactccagct

SEQ ID NO: 108 - DNA, 25 bp, synthetic construct
  source 1..25: mol_type = other DNA
ctcatctcttctgcttctggaactc

SEQ ID NO: 109 - DNA, 22 bp, synthetic construct
  source 1..22: mol_type = other DNA
ttcaggaaacccatcccatgtg

SEQ ID NO: 110 - DNA, 22 bp, synthetic construct
  source 1..22: mol_type = other DNA
atgggtgctcctgtattggttg

SEQ ID NO: 111 - DNA, 25 bp, synthetic construct
  source 1..25: mol_type = other DNA
acaactgtctgatgtcattgaaagg

SEQ ID NO: 112 - DNA, 23 bp, synthetic construct
  source 1..23: mol_type = other DNA
ggaaattgtacagagtcgtgggt

SEQ ID NO: 113 - DNA, 26 bp, synthetic construct
  source 1..26: mol_type = other DNA
actgaagcagaaagctactaaactgt

SEQ ID NO: 114 - DNA, 24 bp, synthetic construct
  source 1..24: mol_type = other DNA
cagatggatacggtttcaaagcca

SEQ ID NO: 115 - DNA, 22 bp, synthetic construct
  source 1..22: mol_type = other DNA
ggcagtatggccatttgacgat

SEQ ID NO: 116 - DNA, 23 bp, synthetic construct
  source 1..23: mol_type = other DNA
gcaacttcagcaaagtctcagga

SEQ ID NO: 117 - DNA, 23 bp, synthetic construct
  source 1..23: mol_type = other DNA
cagtgaatgtagccctttgggat

SEQ ID NO: 118 - DNA, 22 bp, synthetic construct
  source 1..22: mol_type = other DNA
gttccatccatgttgctgcaag

SEQ ID NO: 119 - DNA, 22 bp, synthetic construct
  source 1..22: mol_type = other DNA
acctgctttcacctcatttgct

SEQ ID NO: 120 - DNA, 22 bp, synthetic construct
  source 1..22: mol_type = other DNA
cttccaacaagcacctagggag

SEQ ID NO: 121 - DNA, 20 bp, synthetic construct
  source 1..20: mol_type = other DNA
tccagaaggagacagcaaga

SEQ ID NO: 122 - DNA, 22 bp, synthetic construct
  source 1..22: mol_type = other DNA
actgcttcagaactgagtcaca

SEQ ID NO: 123 - DNA, 24 bp, synthetic construct
  source 1..24: mol_type = other DNA
agttgccttcagagttgagtctag

SEQ ID NO: 124 - DNA, 21 bp, synthetic construct
  source 1..21: mol_type = other DNA
atggtctcgatctgctgacct

SEQ ID NO: 125 - DNA, 22 bp, synthetic construct
  source 1..22: mol_type = other DNA
acaagtacatgggtgcagtgag

SEQ ID NO: 126 - DNA, 23 bp, synthetic construct
  source 1..23: mol_type = other DNA
agtcagacttcctgtcccataga

SEQ ID NO: 127 - DNA, 22 bp, synthetic construct
  source 1..22: mol_type = other DNA
ggagtgaagatggtggagggta

SEQ ID NO: 128 - DNA, 22 bp, synthetic construct
  source 1..22: mol_type = other DNA
atccactgtgaccagactgtga

SEQ ID NO: 129 - DNA, 23 bp, synthetic construct
  source 1..23: mol_type = other DNA
gccaatttctcccatttggaagg

SEQ ID NO: 130 - DNA, 22 bp, synthetic construct
  source 1..22: mol_type = other DNA
accaatcatgccttctcaacgg

SEQ ID NO: 131 - DNA, 26 bp, synthetic construct
  source 1..26: mol_type = other DNA
agaatgacacagatatgttagctgct

SEQ ID NO: 132 - DNA, 25 bp, synthetic construct
  source 1..25: mol_type = other DNA
agctcagttctactgtagtcagaga

SEQ ID NO: 133 - DNA, 22 bp, synthetic construct
  source 1..22: mol_type = other DNA
ggcggaagaattgcttgaactg

SEQ ID NO: 134 - DNA, 24 bp, synthetic construct
  source 1..24: mol_type = other DNA
agctgcctctcctagtctttatgt

SEQ ID NO: 135 - DNA, 21 bp, synthetic construct
  source 1..21: mol_type = other DNA
cgatgctcacgtgtcttcaca

SEQ ID NO: 136 - DNA, 23 bp, synthetic construct
  source 1..23: mol_type = other DNA
ctacttgagtgggctcaatcaca

SEQ ID NO: 137 - DNA, 23 bp, synthetic construct
  source 1..23: mol_type = other DNA
ccaccaaaaactgctcacttctg

SEQ ID NO: 138 - DNA, 24 bp, synthetic construct
  source 1..24: mol_type = other DNA
tctccaacttacagacaggttgag

SEQ ID NO: 139 - DNA, 23 bp, synthetic construct
  source 1..23: mol_type = other DNA
cggaaatgaaaagaactggagca

SEQ ID NO: 140 - DNA, 22 bp, synthetic construct
  source 1..22: mol_type = other DNA
ctgtggtagctccacttgcaat

SEQ ID NO: 141 - DNA, 23 bp, synthetic construct
  source 1..23: mol_type = other DNA
agttaggatgcaacaagaccaga

SEQ ID NO: 142 - DNA, 24 bp, synthetic construct
  source 1..24: mol_type = other DNA
actgttcttcaggcacatagatgt

SEQ ID NO: 143 - DNA, 21 bp, synthetic construct
  source 1..21: mol_type = other DNA
tggctgtacttggccatcttc

SEQ ID NO: 144 - DNA, 22 bp, synthetic construct
  source 1..22: mol_type = other DNA
tagggtggctgaagaaaggaga

SEQ ID NO: 145 - DNA, 22 bp, synthetic construct
  source 1..22: mol_type = other DNA
acctctgtctcaacctcactgt

SEQ ID NO: 146 - DNA, 25 bp, synthetic construct
  source 1..25: mol_type = other DNA
ggttgcttcagtctaagattggatg

(19) United States
(12) Patent Application Publication
(10) Pub. No.: US 2025/0320546 A1
(43) Pub. Date: Oct. 16, 2025

(54) System and Kit for Amplification and Detection Targeting Multiplex DIPs

(71) Applicants: SOUTHERN MEDICAL UNIVERSITY, Guangzhou (CN); GUANGDONG HOMY GENETICS INCORPORATION, Chancheng District, Foshan, Guangdong (CN)

(72) Inventors: Bofeng ZHU, Guangzhou (CN); Fanzhang LEI, Guangzhou (CN); Yangyang ZHENG, Guangzhou (CN); Xiaoqing LIN, Guangzhou (CN); Weian DU, Guangzhou (CN)

(73) Assignees: SOUTHERN MEDICAL UNIVERSITY, Guangzhou (CN); GUANGDONG HOMY GENETICS INCORPORATION, Chancheng District, Foshan, Guangdong (CN)

(21) Appl. No.: 19/000,854

(22) Filed: Dec. 24, 2024

(30) Foreign Application Priority Data
Apr. 11, 2024 (CN) 202410436345.5

(51) Int. Cl. C12Q 1/6844; C12Q 1/6809; C12Q 1/6888

(52) U.S. Cl. CPC C12Q 1/6844; C12Q 1/6809; C12Q 1/6888; C12Q 2600/16; C12Q 2600/166

(57) ABSTRACT

A system for multiplex amplification and detection targeting 73 DIP loci and an application thereof. The system includes a set of primers and an amplification premix composition, wherein the set of primers target each of 73 DIP loci. The present system is capable of simultaneously detecting 73 DIP loci in a single reaction on various types of samples, and thus provides a high-performance novel solution for biogeographical ancestry inference of the five major intercontinental populations including African, European, East Asian, South Asian and Native American populations other than American Mestizos and the intra-East Asian populations including Han Chinese, Southeast Asian and Japanese populations.

### CROSS-REFERENCE TO RELATED APPLICATION

This application is filed on the basis of and claims priorities of Chinese patent application No. 202410436345.5 filed on Apr. 11, 2024, the entire contents of which are incorporated herein by reference.

### INCORPORATION OF SEQUENCE LISTING

The instant application contains a sequence listing which has been submitted electronically in XML format and is hereby incorporated by reference in its entirety. Said XML copy, created on Dec. 23, 2024, is named “P24FS1NW00028US.xml” and is 130,066 bytes in size.

### TECHNICAL FIELD

The present disclosure relates to the field of nucleic acid detection technology, and particularly relates to a system for multiplex amplification and detection targeting 73 DIP (Deletion/Insertion Polymorphism) loci and application thereof.

### BACKGROUND

In the practice of forensic medicine, in addition to common biological samples, various kinds of aged, degraded and other complex biological samples are also omnipresent, which are more difficult to be detected and analyzed. Common forensic DNA analysis of such samples using short tandem repeat markers is prone to DNA typing failure, which brings challenges to forensic identification. In addition, the limited number of short tandem repeat loci that can be accommodated in one reaction system results in a low cumulative identification efficiency of the system, which needs to be supplemented with additional loci to provide more information about the identity of the unknown individual in the case.

Deletion/Insertion Polymorphism (DIP) is a kind of genetic variation, specifically due to the insertion or deletion of DNA fragments. It is widely distributed in the human genome, and is characterized by a low mutation rate (about 10⁻⁸), abundant genetic polymorphisms, short amplicons, absence of artifacts such as stutter peak, and ease of genotyping, which makes it more advantageous in analyzing complex biological samples such as aged and degraded samples. Therefore, DIP marker shows good prospects for forensic applications and is one of the commonly used genetic markers for ancestry inference. The detection of DIP marker is applicable to both the next-generation sequencing platform and the capillary electrophoresis (CE) platform. The main advantage of the former is the high throughput and the ability to detect different types of genetic markers. However, its technical requirements for experimental operators are high and the data processing is relatively complex, coupled with the inevitable sequencing errors, the mutations in the primer binding region and other factors that may easily lead to misjudgment of the results, so it has not yet been routinely used in DNA testing of forensic cases. The traditional PCR-CE platform is highly accurate, low in cost, short time-consuming, and easy to operate, making it a reliable, cost-effective, and efficient detection platform suitable for forensic identification and biogeographical ancestry inference. With the introduction of the six-dye fluorescence detection protocol in 2014, the number of loci capable of simultaneous capillary separation has been greatly increased, and the corresponding system efficacy has been enhanced, making multi-regional and multi-level ancestry inference possible, and contributing to the promotion and application of the DIP panels for biogeographical ancestry inference in grassroots forensic DNA laboratories.

Over the past five years, a series of multiplex amplification systems based on ancestry-informative DIP markers have been developed and validated, such as the Investigator® DIPplex system based on 30 DIP loci, the AGCU InDel 60 kit targeting 60 DIP loci, and the 39 biallelic DIP panel and the 41 multi-InDel panel constructed by Zhu et al. The above systems basically achieve the accurate estimation among the three major ancestral origins (i.e., the African, European, and East Asian), but the effectiveness of the distinction among European, South Asian, and South American individuals still needs to be improved. To further improve the efficacy of the ancestry-informative DIP (AI-DIP) multiplex amplification system in the biogeographical ancestry inference of Asian populations, Sun et al. initially achieved a triple classification of East Asian population, Southeast Asian population, and the Russian Adyghe population in Northeast Asian using 15 multi-InDel loci. Zhang et al, on the other hand, achieved triple classification of 10 Asian populations using 21 efficient AI-DIP loci, and the cross-validation results showed an average accuracy rate of 81.70%.

Although the above studies have initially explored ancestry inference strategy for pan-Asian populations, the number of selected loci and the efficacy of biogeographical ancestry inference are still insufficient to provide more biogeographical information for intra-East Asian populations in forensic practice. Despite the multi-ethnic, multi-cultural and multi-lingual dynamics resulting from the long history of migration and exchange among East Asian populations, previous studies in population molecular biology and phylogenetics suggest a high degree of genetic homogeneity within each ethnic group in East Asia. Therefore, the construction of the biogeographical ancestry inference system for intra-East Asian populations containing more high-performance AI-DIP loci is one of the major challenges in ancestry inference, which puts higher demands on the development technology of the multiplex amplification system, the selection of genetic markers, and the ability of evidence analyses.

The development of the six-dye fluorescent labelling technology has enabled the multiplex amplification system to accommodate a greater number of different types of loci, thus enabling the system to provide more diverse genetic information and improved efficiency of forensic identification. However, as the number of loci in a multiplex amplification system increases, the relative balance among the loci becomes more difficult to control due to their competition, and higher demand is placed on the suitability of amplification condition. Therefore, it is necessary to repeatedly validate the amplification parameters and adjust the primer concentrations and ratios to improve the balance of amplification. In addition, machine learning algorithms have their unique advantages in the selection of genetic markers and evidence analyses, and their applicability to high-dimensional and sparse data helps to mine loci with potential for ancestry inference from massive genome-wide data, but the machine learning methodologies applicable to AI-DIP systems and their efficacy in biogeographical ancestry inference have not yet been developed and validated.

### SUMMARY OF THE INVENTION

The object of the present disclosure is to provide a system for multiplex amplification and detection targeting 73 DIP loci and an application thereof, in order to solve one or more technical problems in the prior art and to provide at least one beneficial option, or to create conditions for so.

According to a first aspect of the present disclosure, provided is a system for multiplex amplification and detection.

According to a second aspect of the present disclosure, provided is a kit comprising the system for multiplex amplification and detection according to the first aspect of the present disclosure.

According to a third aspect of the present disclosure, provided is an application of the system for multiplex amplification and detection according to the first aspect of the present disclosure or the kit according to the second aspect of the present disclosure in biogeographical ancestry inference.

According to a fourth aspect of the present disclosure, provided is a method for biogeographical ancestry inference, comprising using the system for multiplex amplification and detection according to the first aspect of the present disclosure or the kit according to the second aspect of the present disclosure.

The system for multiplex amplification and detection according to the first aspect of the present disclosure comprises a set of primers and an amplification premix composition, wherein the set of primers target 73 DIP loci respectively, including rs73611618, rs28741387, rs141511864, rs71377077, rs10660476, rs879841278, rs55681325, rs140698686, rs200216987, rs71879919, rs10531408, rs59369367, rs56120126, rs71097946, rs77514652, rs561904853, rs5780349, rs2067285, rs5789056, rs5789729, rs141160384, rs139988800, rs141928144, rs56968651, rs59127488, rs1347535145, rs551883542, rs3994057, rs1342356747, rs3044086, rs35450593, rs72104851, rs59005026, rs71712626, rs138600078, rs879662430, rs10564190, rs140202531, rs10573591, rs10630253, rs77624782, rs141471313, rs10600917, rs71408252, rs1404627509, rs74816196, rs112473811, rs57051438, rs766586871, rs10628367, rs58227077, rs143267128, rs140671911, rs138465422, rs200935491, rs141613931, rs66462883, rs71110898, rs35991174, rs35880452, rs59377169, rs778835021, rs79710335, rs59605350, rs112524265, rs5886296, rs59218555, rs67579111, rs71004215, rs56358449, rs140200174, rs56783915, and rs141047228.

Based on genome-wide data from 2598 individuals in the 1000 Genome Project phase III (1KGP) extended dataset, more than 27,000 biallelic DIP loci with potential for biogeographical ancestry inference in major intercontinental and intra-East Asian populations are screened according to a series of locus evaluation criteria, such as insertion length of 2-8 bps. Subsequently, 157 candidate DIP loci are screened by different visualization methods of dimensionality reduction in combination with feature selection algorithms by machine learning, and 73 high-resolution autosomal DIP loci with good detection results are further screened through primer design and evaluations on feature importance. Based on the 73 DIP loci obtained as a set of candidate loci, the system for multiplex amplification and detection according to the present disclosure is designed and developed, which is capable of simultaneously detecting 73 DIP loci in one reaction. The specific technology roadmap is shown in FIG. 1.

In order to achieve one-tube multiplex amplification, in some embodiments according to the first aspect of the present disclosure, the set of primers may contain 73 primer pairs targeting each of the above 73 DIP loci. The nucleotide sequences of each particular primer are shown in Table 1.

In order to improve the accuracy of the multiplex amplification and detection, in some embodiments according to the first aspect of the present disclosure, each of the primer pairs may has a working concentration as shown in Table 1.

In some embodiments according to the first aspect of the present disclosure, at least one primer of each primer pair is labelled at 5′ terminal with a fluorescent dye selected from the group consisting of FAM, HEX, SUM, LYN, PUR, A514, TAMRA, ROX, VIC, A555, PET, NED, TAZ, A488, SF488 and A568. With different fluorescent dyes, the number of amplification products with similar length is converted into fluorescent signal output, thus enabling the detection of multiple DIP loci by the multiplex amplification in a single tube.

In some embodiments according to the first aspect of the present disclosure, primer pairs for the amplification products with large length difference are assigned to the same cluster, and primer pairs for the amplification products with minor length difference are assigned to different clusters. Specifically, the primers with sequences as shown in SEQ ID No:1 to SEQ ID No:30 are set as cluster I, the primers with sequences as shown in SEQ ID No:31 to SEQ ID No:62 are set as cluster II, the primers with sequences as shown in SEQ ID No:63 to SEQ ID No:88 are set as cluster III, the primers with sequences as shown in SEQ ID No:89 to SEQ ID No:118 are set as cluster IV, and the primers with sequences as shown in SEQ ID No:119 to SEQ ID No:146 are set as cluster V. One fluorescent dye is selected for each cluster, and the fluorescent dyes selected for each cluster are different from each other. By assigning the loci to different clusters, the number of fluorescent dyes used can be greatly reduced, thus reducing the difficulty of detection and analysis.

In some embodiments according to the first aspect of the present disclosure, the fluorescent dye selected for the cluster I is FAM, the fluorescent dye selected for the cluster II is HEX, the fluorescent dye selected for the cluster III is SUM, the fluorescent dye selected for the cluster IV is LYN, and the fluorescent dye selected for the cluster V is PUR, as shown in Table 1.

TABLE 1
Nucleotide sequences and working concentrations of 73 primer pairs

| | | | | Working |
|---|---|---|---|---|
| | | SEQ ID | Fluorescent | concentration |
| Locus | Primer sequence (5′→3′) | No | dye | (μmol/L) |
| rs73611618 | AAGGCCCCTTCCTGTGACT | 1 | FAM | 0.0476 |
| | TTGCTCAGGGCCACTTCCA | 2 | | |
| rs28741387 | GCAGTGAGCCAGGATGGTG | 3 | FAM | 0.0311 |
| | GCAGGAATTGCACAATTGCACA | 4 | | |
| rs141511864 | CACCTGCAGTCTTAAGACACCT | 5 | FAM | 0.0385 |
| | CTGATGGCAGGTAAGGTGAGTT | 6 | | |
| rs71377077 | TCTCTTCTCCAGACTGCAAGGA | 7 | FAM | 0.0476 |
| | TGTGGAAGCTTTGGATCCACTC | 8 | | |
| rs10660476 | TGTTCCATTGATCTATGTGCCTGT | 9 | FAM | 0.0458 |
| | CCTGAATAGCCAAGGCCATCC | 10 | | |
| rs879841278 | CAGGCTGGTGAAACCTGATAG | 11 | FAM | 0.0641 |
| | GGTTAGACCCTAGTCTCTCGTT | 12 | | |
| rs55681325 | ACAGTCTCTGTGACACTGACCA | 13 | FAM | 0.0348 |
| | TGCTCTCTGCTGATTTGACAGG | 14 | | |
| rs140698686 | CCATCCCCGACCTAGATTTTCAG | 15 | FAM | 0.0660 |
| | CTGTACTCTGGCCTATGTGACAG | 16 | | |
| rs200216987 | ACAAGAAGGCAAGGTAGACGAAT | 17 | FAM | 0.0513 |
| | TGGTCCAGAAGGTCTAATCAATCA | 18 | | |
| rs71879919 | CTGGAACAAAATAGGTGGTGAGGTA | 19 | FAM | 0.0861 |
| | CCAAAGACAGATATTGCATCTCCCA | 20 | | |
| rs10531408 | AGAAGTCCTTGCTATTGCTCTGAG | 21 | FAM | 0.0531 |
| | TCCAGCTTGGACAAGAGCAGAA | 22 | | |
| rs59369367 | ACCCACACTTGTTTACCCAAGG | 23 | FAM | 0.0586 |
| | GACTGTCTGATGCCAAAGCCTA | 24 | | |
| rs56120126 | GCTCAATAATTCTACAGGGGTCTGT | 25 | FAM | 0.0531 |
| | GCTTTTCTCTGTTTTCCCATTTTCAC | 26 | | |
| rs71097946 | TGCTCCTGGGAAGTTATAAAGGTATTTAA | 27 | FAM | 0.0257 |
| | TGAGGCATTTTATACCTTAGCATGGATTTA | 28 | | |
| | TAT | | | |
| rs77514652 | TGTGACTAGTGGCTACTGTACCA | 29 | FAM | 0.0403 |
| | GAAGGCAAAGGTCAGACCTCTTT | 30 | | |
| rs561904853 | CGAGCACACACAGACACACA | 31 | HEX | 0.0660 |
| | CTCTGTCTCTCTCTGGCTCGT | 32 | | |
| rs5780349 | TCTAGGATTTTCCCCACCCTCT | 33 | HEX | 0.0660 |
| | TGAGGGACATGCATGATTCTCC | 34 | | |
| rs2067285 | AACACTCCACAGTCTAGCCTCAG | 35 | HEX | 0.0751 |
| | GACAGAAGGCATTCCATTGAGAGT | 36 | | |
| rs5789056 | GCTAAGGGAACTCATTTCCATCAGA | 37 | HEX | 0.0403 |
| | CAAGCCTCCAAAATGAGGCTCT | 38 | | |
| rs5789729 | TTACACAGGTTGGAGCATCTTGGA | 39 | HEX | 0.0623 |
| | ATGAGGCTTTGTGAGGTGTGATTC | 40 | | |
| rs141160384 | AGCCTTCTTCAACGTCTGTATCT | 41 | HEX | 0.0708 |
| | CTTTGAGTGCCAACATCTATCTTC | 42 | | |
| rs139988800 | TCAGTGGCATATCCAGGGTCA | 43 | HEX | 0.0793 |
| | ATGGTGCTGGAACAACTGGAC | 44 | | |
| rs141928144 | GAGAGAGAAAGAAAGAAAGGAAAGAAAG | 45 | HEX | 0.0708 |
| | G | | | |
| | CTGTTAGCTATGCTGGTCTCAAGC | 46 | | |
| rs56968651 | ATGACCTCTTCTCTGCCTGGAA | 47 | HEX | 0.0764 |
| | ACTGAGTTCCTGCCTCGAAGTA | 48 | | |
| rs59127488 | CACAGTGCTCAATGCAGCTTC | 49 | HEX | 0.0736 |
| | AAGCTGACAGCCTGGTTACTG | 50 | | |
| rs1347535145 | ACCCCTTCTGCCTACTATTCCA | 51 | HEX | 0.1132 |
| | GAAGGAAGGAAGGAAGGAACGA | 52 | | |
| rs551883542 | CCTGGAAATTGACATTGGCACA | 53 | HEX | 0.0594 |
| | ATGGCTGACCTAAGGCCTAAGA | 54 | | |
| rs3994057 | TGGGTAGAGGGCAGTAAAGTTG | 55 | HEX | 0.0906 |
| | GAAGGGTGTTTACGCCTGTAGA | 56 | | |
| rs1342356747 | GAAGAAAATATTTGTAAACCATGTATCCGA | 57 | HEX | 0.0849 |
| | TG | | | |
| | GTCTTTTTGGATAGTGATCTAGCTAAGAGA | 58 | | |
| | T | | | |
| rs3044086 | CGACAGAGTGGACCTTGTCTC | 59 | HEX | 0.0708 |
| | TGCTGCCCAAAACAGATCCA | 60 | | |
| rs35450593 | GGGCATCTGCAAAAATCCTACAG | 61 | HEX | 0.0531 |
| | ACCTGTGACTCGCTAAACTTATTTAC | 62 | | |
| rs72104851 | AAAACCTTGTGTGGTTGGCATG | 63 | SUM | 0.0403 |
| | CTAGTGCAGTGGCACAGTTCA | 64 | | |
| rs59005026 | CCCTTCCCTTCCTCTTTCTCTTC | 65 | SUM | 0.0476 |
| | GTTCTTTTGTCAGCCCTCACCT | 66 | | |
| rs71712626 | AGCGATAAGAGGGAAACTGGGTA | 67 | SUM | 0.0311 |
| | GCCAGGAATATTCTGTAGGATGCT | 68 | | |
| rs138600078 | AACACATCAGTCAGCAACAGGT | 69 | SUM | 0.0366 |
| | TAGCAACTCAGGAGGCTGAGAT | 70 | | |
| rs879662430 | ATCACAAGATGGTCTGGAAGAAGA | 71 | SUM | 0.1026 |
| | AGGTTGCAGTGAGGTGAGATTG | 72 | | |
| rs10564190 | GCACTCACCCAGATGATTGCTT | 73 | SUM | 0.0879 |
| | GTTCCACTGGAACCACGTAACA | 74 | | |
| rs140202531 | GATCAGGAATGCAAATGCACACA | 75 | SUM | 0.0458 |
| | GAGTTGACCGACAAGTCTTGGT | 76 | | |
| rs10573591 | GATCCCTGTTCTTGCACTTGCT | 77 | SUM | 0.0678 |
| | GTGACTGATGCTGAGTTCCTGG | 78 | | |
| rs10630253 | ATGGCCTTTCTGACCCTACCTT | 79 | SUM | 0.0898 |
| | ACCAGCTGAATTTCCCAGTCTG | 80 | | |
| rs77624782 | CTGAAACTCTTTCTCACCCCCTT | 81 | SUM | 0.0989 |
| | AGAGTCACAGTAAATGTTACAGAACTT | 82 | | |
| rs141471313 | GGCACTTTGTAAGCTGCAACG | 83 | SUM | 0.1869 |
| | AGCACAGTCATATATGTGAGTGCC | 84 | | |
| rs10600917 | ACTAGGTAGGAGTTCTAGGTTCTAAGTG | 85 | SUM | 0.0733 |
| | GGTTCAAAATAAGACCCAGCACAATAG | 86 | | |
| rs71408252 | TGCCCCAAATGCTTATCTTTGAG | 87 | SUM | 0.0421 |
| | CTTGAACCCAAGAGGCGTAAGT | 88 | | |
| rs1404627509 | ATATTGACCAGGCCTAGGGAGT | 89 | LYN | 0.0293 |
| | TACGCACAAACACATGTCGGA | 90 | | |
| rs74816196 | ATCATATAACATCCTGTCCAAGCC | 91 | LYN | 0.0421 |
| | GACGTTTCTGTAAATGCTGAACTC | 92 | | |
| rs112473811 | AGATCCAACAACAGCTTGCACT | 93 | LYN | 0.0825 |
| | CAAGATGTGAGTTCCCTTGGTCT | 94 | | |
| rs57051438 | ACTCCAGGCCAAATGAAATTGC | 95 | LYN | 0.1154 |
| | CAGATCCTGAGATATGTGGAAAGA | 96 | | |
| rs766586871 | GGCAGGAGAATCTCGCTTTAACTC | 97 | LYN | 0.0679 |
| | CTGAGAGACTCAAAGCTTTGAGTGT | 98 | | |
| rs10628367 | CTCATAGAGTTACCTTTCACGCACA | 99 | LYN | 0.0679 |
| | AGCAGTTTCACAGGATTAATGAGTCT | 100 | | |
| rs58227077 | CTATTGGGAGAGGCTGGCTTTG | 101 | LYN | 0.1274 |
| | CCAAGATGTTGATAGGAGGAAGTT | 102 | | |
| rs143267128 | CCAAGTTGCGTCTGGTTTAACTG | 103 | LYN | 0.0764 |
| | TCCTTAATGTCCCACTGGGCTA | 104 | | |
| rs140671911 | CTGTTTTGCCTTGTTGAGAGGTTG | 105 | LYN | 0.0849 |
| | TCCTTGAAAACTACACTTGCATAAGG | 106 | | |
| rs138465422 | TGACAAGAGCAAAACTCCAGCT | 107 | LYN | 0.0340 |
| | CTCATCTCTTCTGCTTCTGGAACTC | 108 | | |
| rs200935491 | TTCAGGAAACCCATCCCATGTG | 109 | LYN | 0.0679 |
| | ATGGGTGCTCCTGTATTGGTTG | 110 | | |
| rs141613931 | ACAACTGTCTGATGTCATTGAAAGG | 111 | LYN | 0.1076 |
| | GGAAATTGTACAGAGTCGTGGGT | 112 | | |
| rs66462883 | ACTGAAGCAGAAAGCTACTAAACTGT | 113 | LYN | 0.0736 |
| | CAGATGGATACGGTTTCAAAGCCA | 114 | | |
| rs71110898 | GGCAGTATGGCCATTTGACGAT | 115 | LYN | 0.1104 |
| | GCAACTTCAGCAAAGTCTCAGGA | 116 | | |
| rs35991174 | CAGTGAATGTAGCCCTTTGGGAT | 117 | LYN | 0.0651 |
| | GTTCCATCCATGTTGCTGCAAG | 118 | | |
| rs35880452 | ACCTGCTTTCACCTCATTTGCT | 119 | PUR | 0.0708 |
| | CTTCCAACAAGCACCTAGGGAG | 120 | | |
| rs59377169 | TCCAGAAGGAGACAGCAAGA | 121 | PUR | 0.0758 |
| | ACTGCTTCAGAACTGAGTCACA | 122 | | |
| rs778835021 | AGTTGCCTTCAGAGTTGAGTCTAG | 123 | PUR | 0.0884 |
| | ATGGTCTCGATCTGCTGACCT | 124 | | |
| rs79710335 | ACAAGTACATGGGTGCAGTGAG | 125 | PUR | 0.0455 |
| | AGTCAGACTTCCTGTCCCATAGA | 126 | | |
| rs59605350 | GGAGTGAAGATGGTGGAGGGTA | 127 | PUR | 0.0758 |
| | ATCCACTGTGACCAGACTGTGA | 128 | | |
| rs112524265 | GCCAATTTCTCCCATTTGGAAGG | 129 | PUR | 0.0379 |
| | ACCAATCATGCCTTCTCAACGG | 130 | | |
| rs5886296 | AGAATGACACAGATATGTTAGCTGCT | 131 | PUR | 0.0303 |
| | AGCTCAGTTCTACTGTAGTCAGAGA | 132 | | |
| rs59218555 | GGCGGAAGAATTGCTTGAACTG | 133 | PUR | 0.0425 |
| | AGCTGCCTCTCCTAGTCTTTATGT | 134 | | |
| rs67579111 | CGATGCTCACGTGTCTTCACA | 135 | PUR | 0.0474 |
| | CTACTTGAGTGGGCTCAATCACA | 136 | | |
| rs71004215 | CCACCAAAAACTGCTCACTTCTG | 137 | PUR | 0.0278 |
| | TCTCCAACTTACAGACAGGTTGAG | 138 | | |
| rs56358449 | CGGAAATGAAAAGAACTGGAGCA | 139 | PUR | 0.0360 |
| | CTGTGGTAGCTCCACTTGCAAT | 140 | | |
| rs140200174 | AGTTAGGATGCAACAAGACCAGA | 141 | PUR | 0.0343 |
| | ACTGTTCTTCAGGCACATAGATGT | 142 | | |
| rs56783915 | TGGCTGTACTTGGCCATCTTC | 143 | PUR | 0.0425 |
| | TAGGGTGGCTGAAGAAAGGAGA | 144 | | |
| rs141047228 | ACCTCTGTCTCAACCTCACTGT | 145 | PUR | 0.0409 |
| | GGTTGCTTCAGTCTAAGATTGGATG | 146 | | |

In some embodiments according to the first aspect of the present disclosure, the components of the amplification premix composition (Master Mix) comprise dNTPs, Taq DNA Polymerase, Tris-HCl buffer, KCl, MgCl₂, and bovine serum albumin (BSA).

The system according to the present disclosure may be used for amplification and detection on human biological samples, which may be genomic DNA extracted from human body fluid/tissue (e.g. blood, saliva, buccal swab, hair with hair follicle, semen, muscle tissue, exfoliated cells, and the like), for example, by Chelex-100, phenol-chloroform, magnetic beads and the like. The system can also be used for direct amplification on various samples without extraction, including blood on filter paper, blood-soaked gauze, FTA cards, saliva, exfoliated cells, and the like.

The kit according to the second aspect of the present disclosure comprises the system for multiplex amplification and detection according to the first aspect of the present disclosure.

In some embodiments according to the second aspect of the present disclosure, the kit may further comprise a control standard for analyses. The control standard may comprises a positive quality control. The positive quality may be an allelic ladder prepared by molecular cloning technique. The positive quality control may comprises a mixture of products from the respective amplification of the corresponding allelic fragments at each locus.

In some embodiments according to the second aspect of the present disclosure, the kit may further comprise a negative quality control. The negative quality control may be nuclease-free water.

In some embodiments according to the third or fourth aspect of the present disclosure, the application of biogeographical ancestry inference comprises individual identification, parentage testing and detection of degraded biomaterials.

In some embodiments according to the third or fourth aspect of the present disclosure, provided is a method for forensic biogeographical ancestry inference and differentiation of a sample from a subject among the African, European, East Asian, South Asian and South American populations, or Han Chinese, Southeast Asian and Japanese populations, comprising using the system for multiplex amplification and detection according to the first aspect of the present disclosure or the kit according to the second aspect of the present disclosure.

In some embodiments according to the second aspect of the present disclosure, the method or the application method specially comprises performing nucleic acid amplification on a sample from a subject using the system or the kit, to obtain an amplification product. Further, the method or the application method specially comprises subjecting the amplification product to detection, for example, by capillary electrophoresis on a genetic analyzer. Further, the method or the application method specially comprises inferring the biogeographical ancestry of the subject according to the result of the detection.

Specially, the PCR amplification product (1 μL), deionized formamide (9.5 μL), and SIZE-500 internal lane standard (0.5 μL) are mixed and denatured at 95° C. for 3 minutes, then incubated on ice for 3 minutes, and subjected to capillary electrophoresis on a genetic analyzer (including, but not limited to, the 3100 series, the 3130 series, and the 3500 series genetic analyzer) for genotyping detection of the 73 DIP loci.

It is of great practical significance to successfully develop a precise biogeographical ancestry inference system based on multiplex DIP genetic markers combined with six-dye fluorescent labelling technology and machine learning algorithms. The present disclosure aims to deeply mine and systematically screen DIP molecular genetic markers strongly associated with different continents and geographic regions on a genome-wide scale by using machine learning algorithms, and to construct a multiplex DIP detection system that targets more high-performance loci based on capillary electrophoresis platform, so as to provide a new detection solution and an evidence analysis process for the precise identification of the biogeographical ancestry of intercontinental and East Asian populations.

The present disclosure has the following advantages:

The present disclosure provides a high-performance system for amplification and detection targeting multiplex AI-DIP, which is applicable for major intercontinental and intra-East Asian populations and is capable of simultaneously detecting 73 DIP loci in a single reaction on various types of samples. The system for multiplex amplification and detection, and the corresponding detection kit and method can simultaneously achieve precise biogeographical ancestry inference of the five major intercontinental populations including African, European, East Asian, South Asian and Native American populations other than American Mestizos. Furthermore, through the system, kit and method according to the present disclosure, East Asian populations can be further subdivided into Han Chinese, Southeast Asian and Japanese populations, which makes up for the shortcoming of the pre-existing systems that has a too large scope of biogeographical ancestry inference, and also effectively enhances the effectiveness of the ancestry inference of intra-East Asian populations, thereby improving the applicability and feasibility of the system in forensic practice.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the technology roadmap for the development of the 73 AI-DIPs multiplex amplification system according to Example 1.

FIG. 2 shows the layout of the amplification products of the 73 DIP loci of the multiplex amplification detection system according to Example 1.

FIG. 3 shows the electrophoretic profile of the allelic ladder according to Example 2.

FIG. 4 shows the electrophoretic profile of the human DNA sample according to Example 3.

FIG. 5 shows the results of the t-SNE dimensionality reduction of the five intercontinental reference populations according to Example 4; wherein, HAN represents Han Chinese, JPT represents Japanese, S-EAS represents Southeast Asian, and RES represents the rest populations; and AFR represents Africa, AMR represents America, EAS represents East Asia, EUR represents Europe, and SAS represents South Asia.

FIG. 6 shows the results of the phylogenetic reconstruction of the five intercontinental reference populations according to Example 4.

FIG. 7 shows the results of the ancestral component analysis of the five intercontinental reference populations according to Example 4.

FIG. 8 shows the results of the ancestral component analysis of the East Asian reference populations according to Example 4

FIG. 9 shows the 10-fold cross-validation results of the biogeographical ancestry inference according to Example 4, wherein panel (A) shows the standardized 10-fold cross-validation confusion matrices for a range of biogeographical ancestry inference models constructed based on intercontinental reference populations from five continents, and panel (B) shows the standardized 10-fold cross-validation confusion matrices for biogeographical ancestry inference models constructed based on East Asian reference populations; wherein, HAN represents Han Chinese, JPT represents Japanese, S-EAS represents Southeast Asian, and RES represents the rest populations; and AFR represents Africa, AMR represents America, EAS represents East Asia, EUR represents Europe, and SAS represents South Asia.

### DETAILED DESCRIPTION

The technical solutions of the present disclosure are further described by the following examples. Those skilled in the art should understand that the Examples are described for only helping understanding the present application, and should not be regarded as any specific limitation to the present application. Modifications and substitutions made to the methods, steps or conditions of the disclosure without departing from the spirit and substance of the disclosure are within the scope of the disclosure.

Unless specifically specified, the raw materials, reagents or devices used in the following examples are commercially available from conventional sources or can be obtained by the prior art methods. Unless specifically specified, the assay or test methods are conventional methods in the art. The methods for molecular biology tests not described in the following examples can be found in the Molecular Cloning: A Laboratory Manual (3rd edition) or according to the kit and product instructions.

### Example 1: AI-DIP Loci Screening and Primer Design for the Multiplex Amplification System

### (1) Screening of 73 DIP Loci

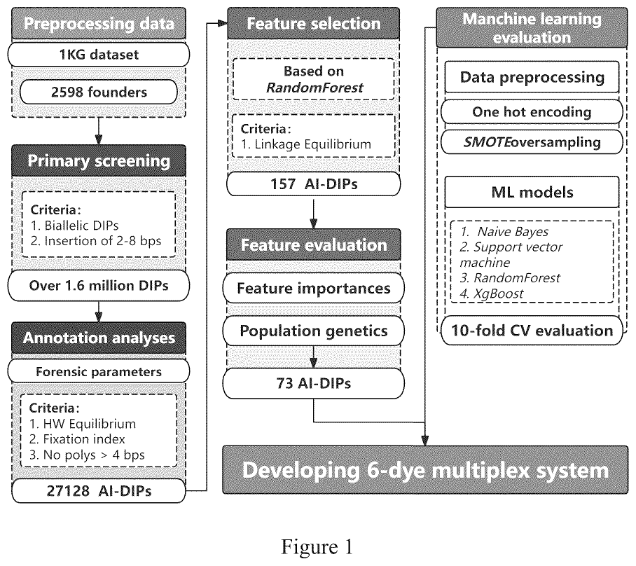

A systematic and comprehensive screening of biogeographical ancestry inference markers was conducted based on genome-wide data from 2598 individuals in the 1KGP extended dataset. The vcf files of DIP loci on 22 pairs of human autosomes were extracted on a genome-wide scale by PLINK software, and the set of candidate loci used to construct the system was finally identified according to the selection criteria of the loci (such as insertion length of 2-8 bps) through data preprocessing, visualization of dimensionality reduction (t-Distributed Stochastic Neighbor Embedding, t-SNE), tree model-based feature importance assessment, and cross-validations, following the specific technology roadmap shown in FIG. 1. The candidate loci were rs73611618, rs28741387, rs141511864, rs71377077, rs10660476, rs879841278, rs55681325, rs140698686, rs200216987, rs71879919, rs10531408, rs59369367, rs56120126, rs71097946, rs77514652, rs561904853, rs5780349, rs2067285, rs5789056, rs5789729, rs141160384, rs139988800, rs141928144, rs56968651, rs59127488, rs1347535145, rs551883542, rs3994057, rs1342356747, rs3044086, rs35450593, rs72104851, rs59005026, rs71712626, rs138600078, rs879662430, rs10564190, rs140202531, rs10573591, rs10630253, rs77624782, rs141471313, rs10600917, rs71408252, rs1404627509, rs74816196, rs112473811, rs57051438, rs766586871, rs10628367, rs58227077, rs143267128, rs140671911, rs138465422, rs200935491, rs141613931, rs66462883, rs71110898, rs35991174, rs35880452, rs59377169, rs778835021, rs79710335, rs59605350, rs112524265, rs5886296, rs59218555, rs67579111, rs71004215, rs56358449, rs140200174, rs56783915, and rs141047228.

### (2) Design of Primer Combination for the Selected DIP Loci

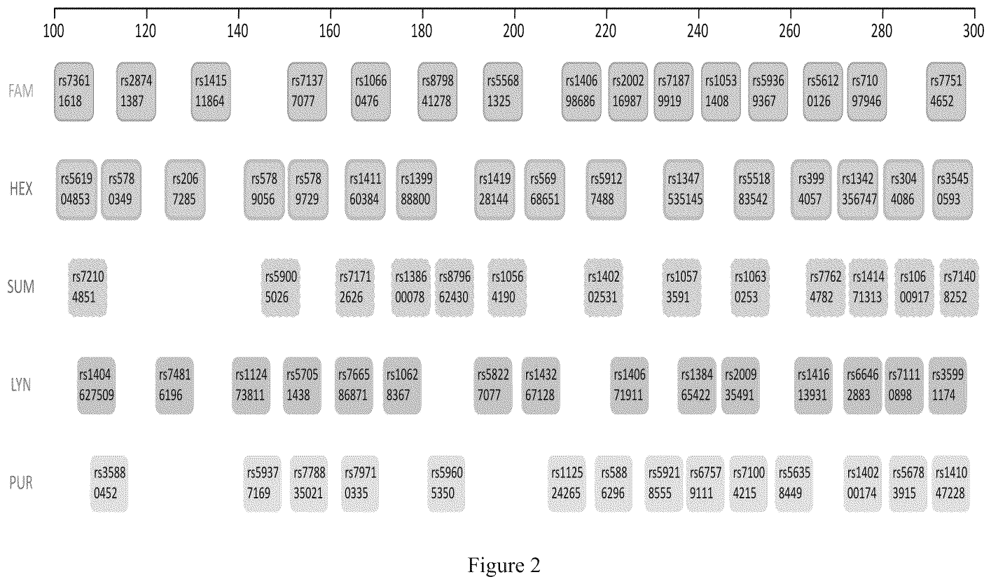

As the fragment sizes of the amplification products and the amplification efficiency of the primers of each polymorphic loci differ greatly, the primer pairs for all loci were repeatedly tested and screened using the multiplex amplification system according to the results of the amplification and detection of a single locus. Further, test and analyses were conducted on a variety of sample types, and at the same time, the concentrations of the primers for each of the loci were optimized and adjusted through a series of experiments to gradually improve the amplification equilibrium of the primer combination, so as to finally obtain the optimal concentrations of the primer pairs in the multiplex amplification detection system. The obtained primer combination are shown in Table 1, and the layout of the corresponding amplification products for all DIP loci is shown in FIG. 2.

### Example 2: Construction of the Multiplex Amplification Detection System

On the basis of the successful establishment of the single-locus amplification system, new primers were gradually added to the amplification system for testing. The parameters of the multiplex amplification reaction, including cycling parameter, annealing temperature, final extension time, enzyme dosage, volume of the multiplex amplification reaction, and amount of the template DNA, were determined through repeated experiments, so as to achieve stable and balanced detection results. The optimal volume of the amplification reaction was finally determined to be 10 μL in this example, and the components and reagent volumes of the multiplex amplification detection system are as shown in Table 2.

TABLE 2
Components and reagent volumes

| Component | Reagent volume |
|---|---|
| Human biological | 0.5-1.2 mm blood spots or saliva spots, or 0.5-2 |
| sample | ng of human genomic DNA obtained by extraction |
| Master Mix | 4 μL |
| Primer mixture | 2 μL |
| Nuclease-free water | add to 10 μL |

The Master Mix contained dNTPs, Taq DNA Polymerase, Tris-HCl buffer, KCl, MgCl₂, and BSA. Human biological samples to be tested was genomic DNA extracted from human body fluid/tissue (e.g., blood, saliva, buccal swab, hair with hair follicle, semen, muscle tissue, exfoliated cells, and the like), and the human DNA was extracted through Chelex-100, phenol-chloroform, or magnetic beads, and quantified. A variety of sample types (e.g., blood on filter paper, blood-soaked gauze, FTA cards, saliva, exfoliated cells, and the like) can also be directly amplified without extraction by the system of this example. The specific multiplex amplification procedure is shown in Table 3.

TABLE 3
Multiplex amplification procedure

| Initial denaturation | Thermal cycling (30 cycles) | Final extension |
|---|---|---|
| 95° C. for 2 minutes | 94° C. for 30 seconds | 72° C. for 10 minutes |
| | 58° C. for 60 seconds | |
| | 72° C. for 50 seconds | |

The amplification products were detected by capillary electrophoresis. Specially, the PCR amplification product (1 μL), deionized formamide (9.5 μL) and SIZE-500 internal lane standard (0.5 μL) were first mixed, then denatured at 95° C. for 3 minutes and incubated on ice for 3 minutes, and subjected to capillary electrophoresis on a genetic analyzer (including but not limited to, the Applied Biosystems® 3100 series, 3130 series, and 3500 series genetic analyzer). After capillary electrophoresis, the data were processed and analyzed using the GeneMapper® ID-X software. First, the corresponding Bin and Panel files were prepared according to the format requirement of the software, and the insertion allele in the DIP marker was named “I” and the deletion allele was named “D”. Subsequently, the capillary electrophoresis data were imported, and the electrophoresis results of the 73 DIP loci were analyzed with the corresponding analytical parameters (e.g., Panel, Bin, Analysis Method, and Size Standard).

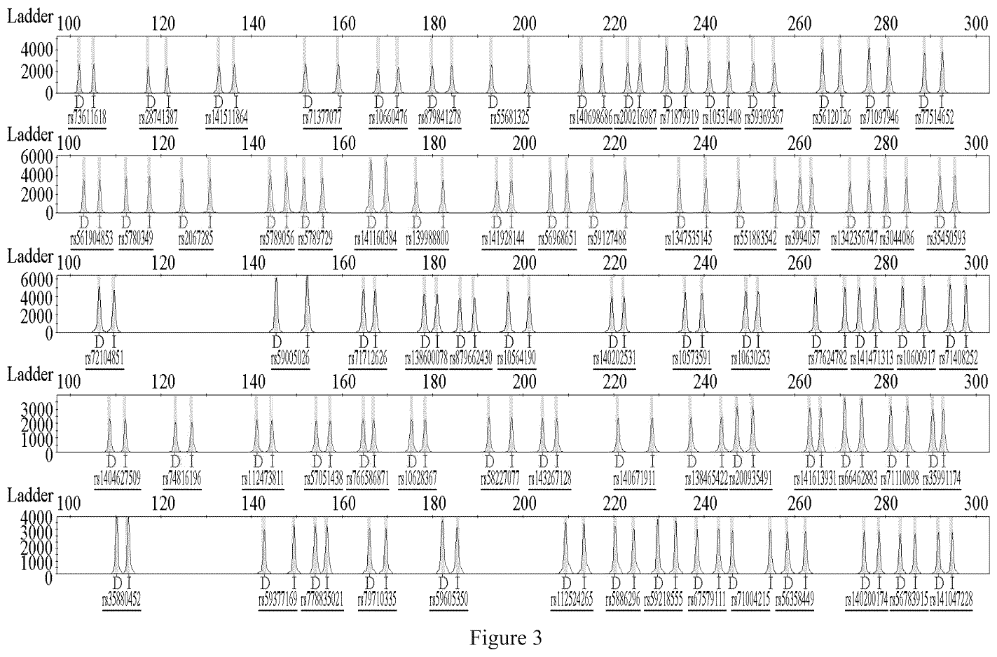

The present disclosure also applied molecular cloning technique to prepare the allelic ladder for data analyses, which consisted of a mixture of amplification products of the corresponding allelic fragments by the primers at each locus. FIG. 3 shows the electrophoretic profile of the allelic ladder of this example.

### Example 3: Practical Application of the Multiplex Amplification Detection System

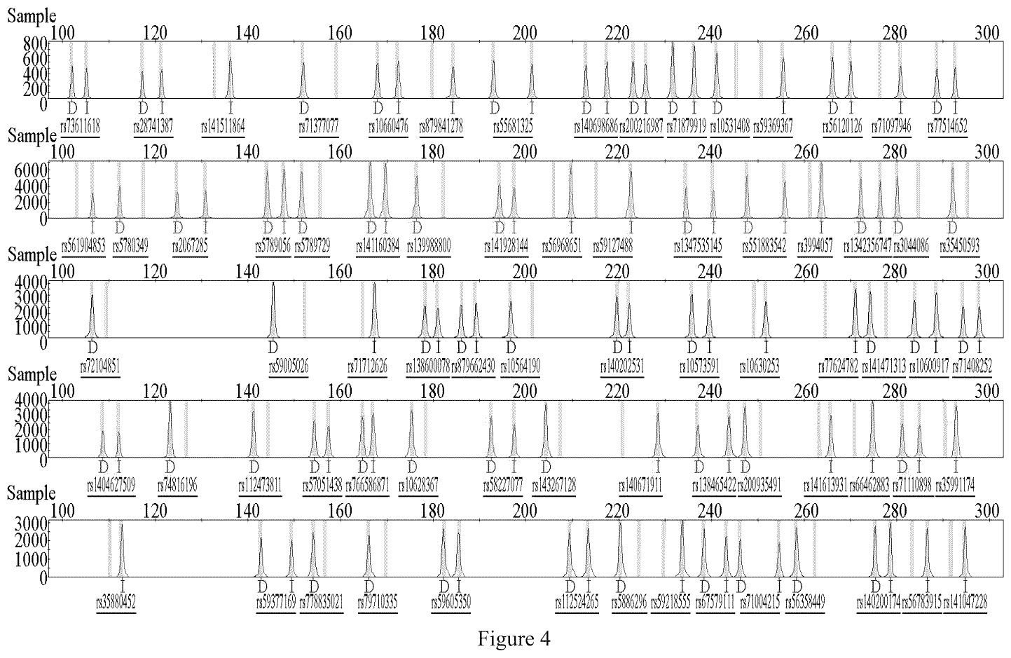

The multiplex amplification detection system provided according to Example 2 was applied in the detection of actual human DNA samples. 1 ng DNA sample of human blood card extracted by Chelex-100 method was detected following the procedure in Example 2. The corresponding electrophoretic profile is shown in FIG. 4. Each locus was accurately detected with relatively balanced peak height, indicating that the above system could achieve effective detection of human DNA samples.

### Example 4: Assessment of the Efficacy of 73 DIP Loci for Biogeographical Ancestry Inference

The efficacy of the selected DIP genetic markers of 73 DIP loci for biogeographical ancestry inference was assessed using intercontinental population data from five continents in the 1KGP extended dataset based on population genetics method such as the algorithm of t-SNE, phylogenetic reconstruction, and ADMIXTURE analysis.

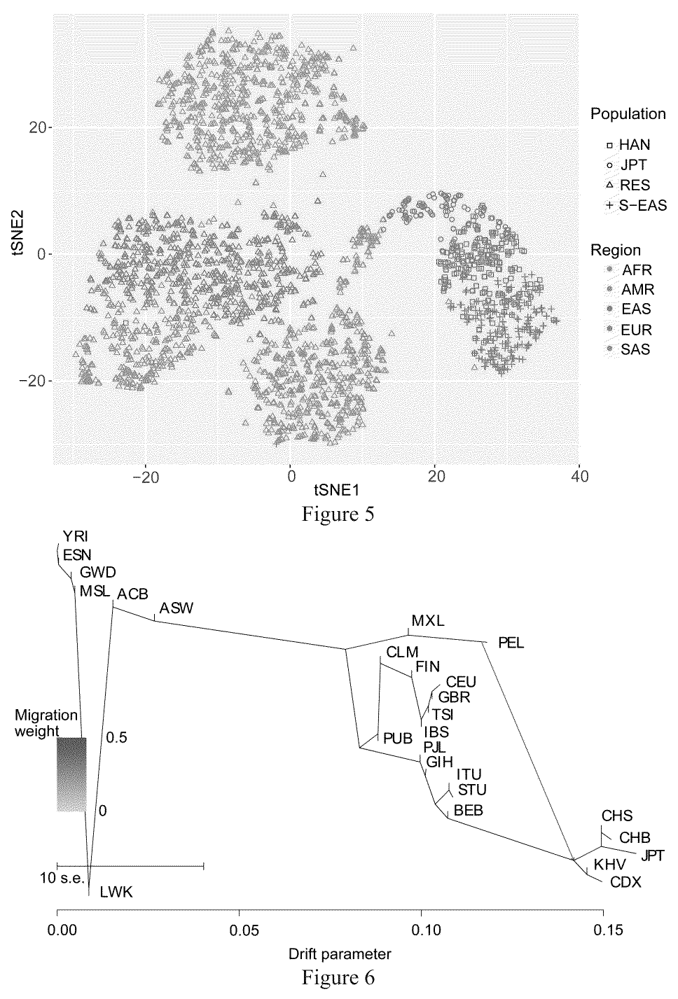

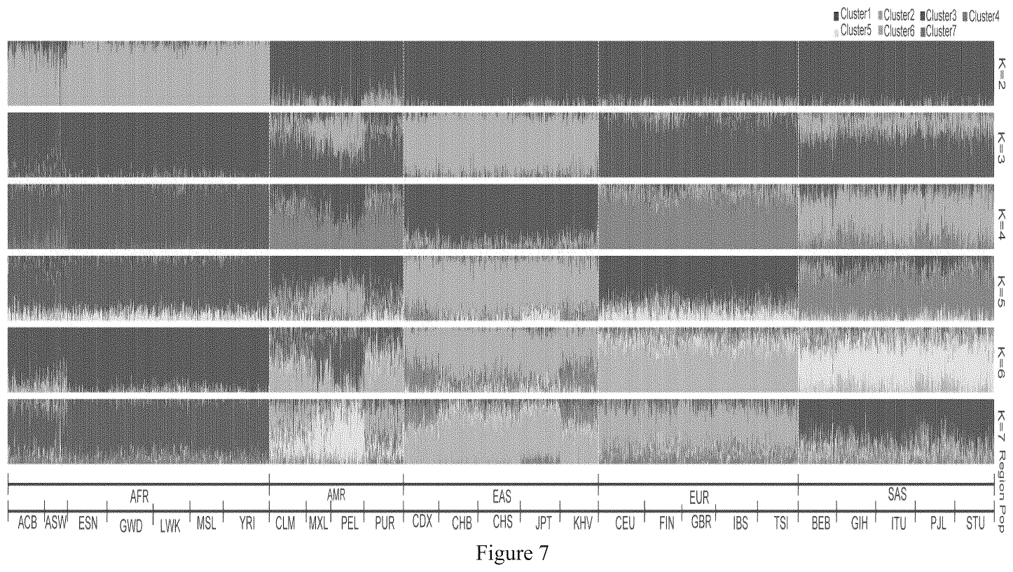

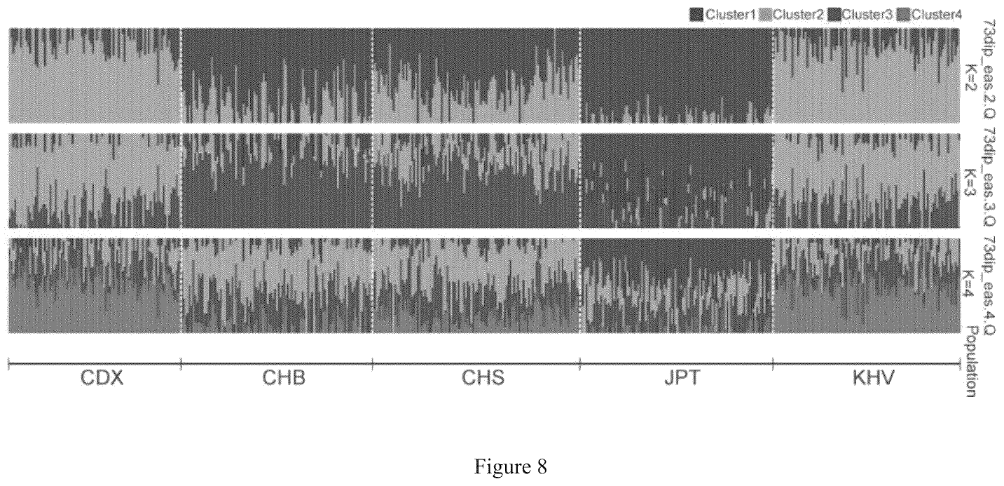

The results of t-SNE showed that the 73 DIP loci selected by the system could basically be used to distinguish the five intercontinental populations, and the preliminary clustering of the Han Chinese (HAN), Japanese (JPT), and Southeast Asian (S-EAS) populations could also be observed (FIG. 5). The results of phylogenetic reconstruction based on the maximum likelihood ratio method clearly showed the evolutionary branching of the five intercontinental populations, and three sub-branches could be seen within the East Asian populations (FIG. 6). Ancestral component analysis based on the ADMIXTURE software showed that the system was able to show the genetic differences among different intercontinental populations at the level of the five intercontinental populations, with an optimal K value of 4 (FIG. 7), and the genetic differences within the East Asian populations could also be identified, with an optimal K value of 2 (FIG. 8).

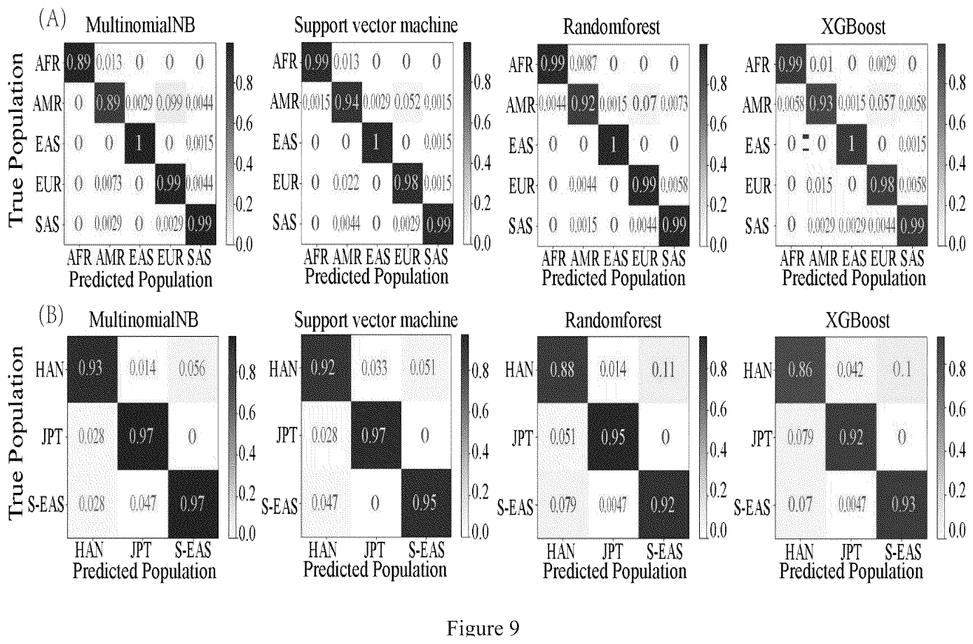

A series of biogeographical ancestry inference models were constructed based on the 73 loci panel described in the present disclosure and 1KGP extended dataset using algorithms such as the multinomial Naive Bayes, support vector machine, random forest, and extreme gradient boosting (XGBoost). The results of the 10-fold cross-validation confusion matrices for the above models showed that the 73 DIP panel described in the present disclosure correctly classified the five intercontinental populations with an average correct rate more than 98% (FIG. 9A), and for the intra-East Asian populations with an average correct rate more than 90% (FIG. 9B). Table 4 shows the 10-fold cross-validation results of each machine learning model for the test set under the classification tasks with different ranges of biogeographical ancestry inference. As shown in Table 4, the 73 DIP loci showed high classification validity and generalization ability among the five intercontinental populations (i.e., Africa, Europe, East Asia, South Asia, and the Americas) and the intra-East Asian populations (i.e., Han Chinese, Japanese, and Southeast Asian populations).

The results of the population genetics analyses described above indicated that the 73 DIP loci contained in this panel had good performance for ancestry inference, and could further subdivide the ancestral origins of intra-East Asian populations.

TABLE 4
10-fold cross-validation results of
biogeographical ancestry inference

| Range for | Machine | Average | | | |
|---|---|---|---|---|---|
| biogeographical | learning | preci- | | Preci- | F1 |
| ancestry inference | model | sion | Recall | sion | score |
| Classification | Multinomial | 0.9751 | 0.9752 | 0.977 | 0.975 |
| of the five | Naive Bayes | | | | |
| intercontinental | Support | 0.9794 | 0.9794 | 0.9803 | 0.9793 |
| populations | vector | | | | |
| | machine | | | | |
| | Random | 0.9797 | 0.9797 | 0.9818 | 0.9884 |
| | forest | | | | |
| | XGBoost | 0.9797 | 0.9797 | 0.9813 | 0.9795 |
| Classification | Multinomial | 0.9258 | 0.9257 | 0.9309 | 0.9244 |
| of the intra- | Naive Bayes | | | | |
| East Asian | Support | 0.9412 | 0.9412 | 0.943 | 0.9412 |
| populations | vector | | | | |
| | machine | | | | |
| | Random | 0.9323 | 0.932 | 0.9322 | 0.9322 |
| | forest | | | | |
| | XGBoost | 0.9048 | 0.9043 | 0.9106 | 0.9047 |

### Example 5: Application Validation of the Multiplex System of the Present Disclosure

According to the method and procedure of Example 2, using the multiplex system according to Example 1, the efficacy of the system for inferring the biogeographical ancestries of actual samples was assessed by genotyping two different individuals of known biogeographic ancestral origins and inferring the biogeographical ancestries of these individuals by the Naive Bayes method, using a 9948 DNA standard as the positive control.

The genotyping results of the two different individuals of known biogeographic ancestral origins are shown in Table 5.

TABLE 5
Genotyping results of the two different individuals

Genotyping of the samples

| Locus | A1 | B1 |
|---|---|---|
| rs73611618 | D | D |
| rs28741387 | I | I |
| rs141511864 | D | D |
| rs71377077 | D | D |
| rs10660476 | D | D |
| rs879841278 | I | I |
| rs55681325 | I | D, I |
| rs140698686 | D, I | I |
| rs200216987 | D | D |
| rs71879919 | I | I |
| rs10531408 | I | I |
| rs59369367 | I | I |
| rs56120126 | I | I |
| rs71097946 | D | D |
| rs77514652 | I | I |
| rs561904853 | I | I |
| rs5780349 | D, I | D, I |
| rs2067285 | D, I | I |
| rs5789056 | D | D |
| rs5789729 | D, I | I |
| rs141160384 | D, I | I |
| rs139988800 | I | I |
| rs141928144 | I | I |
| rs56968651 | I | I |
| rs59127488 | I | I |
| rs1347535145 | D | D |
| rs551883542 | D, I | I |
| rs3994057 | I | I |
| rs1342356747 | D | D |
| rs3044086 | D | D |
| rs35450593 | I | I |
| rs72104851 | I | I |
| rs59005026 | D | D |
| rs71712626 | D | D |
| rs138600078 | I | I |
| rs879662430 | I | I |
| rs10564190 | D, I | I |
| rs140202531 | I | I |
| rs10573591 | I | I |
| rs10630253 | D, I | I |
| rs77624782 | D | D |
| rs141471313 | D, I | D |
| rs10600917 | D, I | I |
| rs71408252 | D, I | I |
| rs1404627509 | D | D |
| rs74816196 | I | D, I |
| rs112473811 | I | I |
| rs57051438 | D | D |
| rs766586871 | I | I |
| rs10628367 | D, I | D |
| rs58227077 | D, I | D |
| rs143267128 | D, I | D, I |
| rs140671911 | I | I |
| rs138465422 | D | D |
| rs200935491 | I | D, I |
| rs141613931 | I | I |
| rs66462883 | D | D |
| rs71110898 | I | I |
| rs35991174 | D, I | I |
| rs35880452 | I | D, I |
| rs59377169 | D | D |
| rs778835021 | D | D |
| rs79710335 | I | D |
| rs59605350 | I | I |
| rs112524265 | I | I |
| rs5886296 | D | D, I |
| rs59218555 | D | D, I |
| rs67579111 | D, I | D |
| rs71004215 | I | D, I |
| rs56358449 | I | I |
| rs140200174 | D, I | I |
| rs56783915 | D, I | D |
| rs141047228 | I | I |

The results of the population matching probability and the likelihood ratio for the two samples based on the 73 DIP loci are shown in Table 6.

TABLE 6
The results of the population matching
probability and likelihood ratio

| | Actual | Predicted | Population | |
|---|---|---|---|---|
| | biogeographic | biogeographic | matching | Likelihood |
| Sample | ancestral origin | ancestral origin | probability | ratio |
| A1 | East Asia | East Asia | 1 | |
| | | Africa | 6.33E−29 | 1.58E+28 |
| | | Americas | 7.27E−11 | 1.37E+10 |
| | | Europe | 3.81E−13 | 2.63E+12 |
| | | South Asia | 1.80E−10 | 5.55E+09 |
| B1 | Han Chinese | Han Chinese | 0.9969 | |
| | | Japanese | 2.83E−03 | 351.68 |
| | | Southeast Asia | 3.01E−04 | 3309.31 |

The above results showed that both 2 actual samples could be detected with complete genotyping based on all loci, and both were correctly inferred to be of actual biogeographic ancestral origins, indicating that the system of the present disclosure was capable of genotyping and inferring the biogeographical ancestries of actual samples with excellent performance, and that the system and method of the present disclosure could be applied to perform effective biogeographical ancestry inference for humans.

It is apparent for those skilled in the art that the present disclosure is not limited to the details of the above examples, and that the present disclosure may be implemented in other specific forms without departing from the technical solution or essential features of the present disclosure. Accordingly, the examples of the present disclosure should be regarded as exemplary and non-limiting, and the scope of the present disclosure is limited by the appended claims and not by the above description, so that the present disclosure is intended to cover all variations within the meaning and scope of the same elements in the claims.

We claim:

1. A system for multiplex amplification and detection, comprising a set of primers and an amplification premix composition, wherein the set of primers target each of 73 DIP loci respectively, and the 73 DIP loci comprise rs73611618, rs28741387, rs141511864, rs71377077, rs10660476, rs879841278, rs55681325, rs140698686, rs200216987, rs71879919, rs10531408, rs59369367, rs56120126, rs71097946, rs77514652, rs561904853, rs5780349, rs2067285, rs5789056, rs5789729, rs141160384, rs139988800, rs141928144, rs56968651, rs59127488, rs1347535145, rs551883542, rs3994057, rs1342356747, rs3044086, rs35450593, rs72104851, rs59005026, rs71712626, rs138600078, rs879662430, rs10564190, rs140202531, rs10573591, rs10630253, rs77624782, rs141471313, rs10600917, rs71408252, rs1404627509, rs74816196, rs112473811, rs57051438, rs766586871, rs10628367, rs58227077, rs143267128, rs140671911, rs138465422, rs200935491, rs141613931, rs66462883, rs71110898, rs35991174, rs35880452, rs59377169, rs778835021, rs79710335, rs59605350, rs112524265, rs5886296, rs59218555, rs67579111, rs71004215, rs56358449, rs140200174, rs56783915, and rs141047228.

2. The system according to claim 1, wherein, the set of primers comprise a primer pair targeting rs73611618 having sequences as shown in SEQ ID No:1 and SEQ ID No:2; a primer pair targeting rs28741387 having sequences as shown in SEQ ID No:3 and SEQ ID No:4; a primer pair targeting rs141511864 having sequences as shown in SEQ ID No:5 and SEQ ID No:6; a primer pair targeting rs71377077 having sequences as shown in SEQ ID No:7 and SEQ ID No:8; a primer pair targeting rs10660476 having sequences as shown in SEQ ID No:9 and SEQ ID No:10; a primer pair targeting rs879841278 having sequences as shown in SEQ ID No:11 and SEQ ID No:12; a primer pair targeting rs55681325 having sequences as shown in SEQ ID No:13 and SEQ ID No:14; a primer pair targeting rs140698686 having sequences as shown in SEQ ID No:15 and SEQ ID No:16; a primer pair targeting rs200216987 having sequences as shown in SEQ ID No:17 and SEQ ID No:18; a primer pair targeting rs71879919 having sequences as shown in SEQ ID No:19 and SEQ ID No:20; a primer pair targeting rs10531408 having sequences as shown in SEQ ID No:21 and SEQ ID No:22; a primer pair targeting rs59369367 having sequences as shown in SEQ ID No:23 and SEQ ID No:24; a primer pair targeting rs56120126 having sequences as shown in SEQ ID No:25 and SEQ ID No:26; a primer pair targeting rs71097946 having sequences as shown in SEQ ID No:27 and SEQ ID No:28; a primer pair targeting rs77514652 having sequences as shown in SEQ ID No:29 and SEQ ID No:30; a primer pair targeting rs561904853 having sequences as shown in SEQ ID No:31 and SEQ ID No:32; a primer pair targeting rs5780349 having sequences as shown in SEQ ID No:33 and SEQ ID No:34; a primer pair targeting rs2067285 having sequences as shown in SEQ ID No:35 and SEQ ID No:36; a primer pair targeting rs5789056 having sequences as shown in SEQ ID No:37 and SEQ ID No:38; a primer pair targeting rs5789729 having sequences as shown in SEQ ID No:39 and SEQ ID No:40; a primer pair targeting rs141160384 having sequences as shown in SEQ ID No:41 and SEQ ID No:42; a primer pair targeting rs139988800 having sequences as shown in SEQ ID No:43 and SEQ ID No:44; a primer pair targeting rs141928144 having sequences as shown in SEQ ID No:45 and SEQ ID No:46; a primer pair targeting rs56968651 having sequences as shown in SEQ ID No:47 and SEQ ID No:48; a primer pair targeting rs59127488 having sequences as shown in SEQ ID No:49 and SEQ ID No:50; a primer pair targeting rs1347535145 having sequences as shown in SEQ ID No:51 and SEQ ID No:52; a primer pair targeting rs551883542 having sequences as shown in SEQ ID No:53 and SEQ ID No:54; a primer pair targeting rs3994057 having sequences as shown in SEQ ID No:55 and SEQ ID No:56; a primer pair targeting rs1342356747 having sequences as shown in SEQ ID No:57 and SEQ ID No:58; a primer pair targeting rs3044086 having sequences as shown in SEQ ID No:59 and SEQ ID No:60; a primer pair targeting rs35450593 having sequences as shown in SEQ ID No:61 and SEQ ID No:62; a primer pair targeting rs72104851 having sequences as shown in SEQ ID No:63 and SEQ ID No:64; a primer pair targeting rs59005026 having sequences as shown in SEQ ID No:65 and SEQ ID No:66; a primer pair targeting rs71712626 having sequences as shown in SEQ ID No:67 and SEQ ID No:68; a primer pair targeting rs138600078 having sequences as shown in SEQ ID No:69 and SEQ ID No:70; a primer pair targeting rs879662430 having sequences as shown in SEQ ID No:71 and SEQ ID No:72; a primer pair targeting rs10564190 having sequences as shown in SEQ ID No:73 and SEQ ID No:74; a primer pair targeting rs140202531 having sequences as shown in SEQ ID No:75 and SEQ ID No:76; a primer pair targeting rs10573591 having sequences as shown in SEQ ID No:77 and SEQ ID No:78; a primer pair targeting rs10630253 having sequences as shown in SEQ ID No:79 and SEQ ID No:80; a primer pair targeting rs77624782 having sequences as shown in SEQ ID No:81 and SEQ ID No:82; a primer pair targeting rs141471313 having sequences as shown in SEQ ID No:83 and SEQ ID No:84; a primer pair targeting rs10600917 having sequences as shown in SEQ ID No:85 and SEQ ID No:86; a primer pair targeting rs71408252 having sequences as shown in SEQ ID No:87 and SEQ ID No:88; a primer pair targeting rs1404627509 having sequences as shown in SEQ ID No:89 and SEQ ID No:90; a primer pair targeting rs74816196 having sequences as shown in SEQ ID No:91 and SEQ ID No:92; a primer pair targeting rs112473811 having sequences as shown in SEQ ID No:93 and SEQ ID No:94; a primer pair targeting rs57051438 having sequences as shown in SEQ ID No:95 and SEQ ID No:96; a primer pair targeting rs766586871 having sequences as shown in SEQ ID No:97 and SEQ ID No:98; a primer pair targeting rs10628367 having sequences as shown in SEQ ID No:99 and SEQ ID No:100; a primer pair targeting rs58227077 having sequences as shown in SEQ ID No:101 and SEQ ID No:102; a primer pair targeting rs143267128 having sequences as shown in SEQ ID No:103 and SEQ ID No:104; a primer pair targeting rs140671911 having sequences as shown in SEQ ID No:105 and SEQ ID No:106; a primer pair targeting rs138465422 having sequences as shown in SEQ ID No:107 and SEQ ID No:108; a primer pair targeting rs200935491 having sequences as shown in SEQ ID No:109 and SEQ ID No:110; a primer pair targeting rs141613931 having sequences as shown in SEQ ID No:111 and SEQ ID No:112; a primer pair targeting rs66462883 having sequences as shown in SEQ ID No:113 and SEQ ID No:114; a primer pair targeting rs71110898 having sequences as shown in SEQ ID No:115 and SEQ ID No:116; a primer pair targeting rs35991174 having sequences as shown in SEQ ID No:117 and SEQ ID No:118; a primer pair targeting rs35880452 having sequences as shown in SEQ ID No:119 and SEQ ID No:120; a primer pair targeting rs59377169 having sequences as shown in SEQ ID No:121 and SEQ ID No:122; a primer pair targeting rs778835021 having sequences as shown in SEQ ID No:123 and SEQ ID No:124; a primer pair targeting rs79710335 having sequences as shown in SEQ ID No:125 and SEQ ID No:126; a primer pair targeting rs59605350 having sequences as shown in SEQ ID No:127 and SEQ ID No:128; a primer pair targeting rs112524265 having sequences as shown in SEQ ID No:129 and SEQ ID No:130; a primer pair targeting rs5886296 having sequences as shown in SEQ ID No:131 and SEQ ID No:132; a primer pair targeting rs59218555 having sequences as shown in SEQ ID No:133 and SEQ ID No:134; a primer pair targeting rs67579111 having sequences as shown in SEQ ID No:135 and SEQ ID No:136; a primer pair targeting rs71004215 having sequences as shown in SEQ ID No:137 and SEQ ID No:138; a primer pair targeting rs56358449 having sequences as shown in SEQ ID No:139 and SEQ ID No:140; a primer pair targeting rs140200174 having sequences as shown in SEQ ID No:141 and SEQ ID No:142; a primer pair targeting rs56783915 having sequences as shown in SEQ ID No:143 and SEQ ID No:144; and a primer pair targeting rs141047228 having sequences as shown in SEQ ID No:145 and SEQ ID No:146.

3. The system according to claim 2, wherein, a working concentration of the primer pair targeting rs73611618 is 0.0476 μmol/L; a working concentration of the primer pair targeting rs28741387 is 0.0311 μmol/L; a working concentration of the primer pair targeting rs141511864 is 0.0385 μmol/L; a working concentration of the primer pair targeting rs71377077 is 0.0476 μmol/L; a working concentration of the primer pair targeting rs10660476 is 0.0458 μmol/L; a working concentration of the primer pair targeting rs879841278 is 0.0641 μmol/L; a working concentration of the primer pair targeting rs55681325 is 0.0348 μmol/L; a working concentration of the primer pair targeting rs140698686 is 0.0660 μmol/L; a working concentration of the primer pair targeting rs200216987 is 0.0513 μmol/L; a working concentration of the primer pair targeting rs71879919 is 0.0861 μmol/L; a working concentration of the primer pair targeting rs10531408 is 0.0531 μmol/L; a working concentration of the primer pair targeting rs59369367 is 0.0586 μmol/L; a working concentration of the primer pair targeting rs56120126 is 0.0531 μmol/L; a working concentration of the primer pair targeting rs71097946 is 0.0257 μmol/L; a working concentration of the primer pair targeting rs77514652 is 0.0403 μmol/L; a working concentration of the primer pair targeting rs561904853 is 0.0660 μmol/L; a working concentration of the primer pair targeting rs5780349 is 0.0660 μmol/L; a working concentration of the primer pair targeting rs2067285 is 0.0751 μmol/L; a working concentration of the primer pair targeting rs5789056 is 0.0403 μmol/L; a working concentration of the primer pair targeting rs5789729 is 0.0623 μmol/L; a working concentration of the primer pair targeting rs141160384 is 0.0708 μmol/L; a working concentration of the primer pair targeting rs139988800 is 0.0793 μmol/L; a working concentration of the primer pair targeting rs141928144 is 0.0708 μmol/L; a working concentration of the primer pair targeting rs56968651 is 0.0764 μmol/L; a working concentration of the primer pair targeting rs59127488 is 0.0736 μmol/L; a working concentration of the primer pair targeting rs1347535145 is 0.1132 μmol/L; a working concentration of the primer pair targeting rs551883542 is 0.0594 μmol/L; a working concentration of the primer pair targeting rs3994057 is 0.0906 μmol/L; a working concentration of the primer pair targeting rs1342356747 is 0.0849 μmol/L; a working concentration of the primer pair targeting rs3044086 is 0.0708 μmol/L; a working concentration of the primer pair targeting rs35450593 is 0.0531 μmol/L; a working concentration of the primer pair targeting rs72104851 is 0.0403 μmol/L; a working concentration of the primer pair targeting rs59005026 is 0.0476 μmol/L; a working concentration of the primer pair targeting rs71712626 is 0.0311 μmol/L; a working concentration of the primer pair targeting rs138600078 is 0.0366 μmol/L; a working concentration of the primer pair targeting rs879662430 is 0.1026 μmol/L; a working concentration of the primer pair targeting rs10564190 is 0.0879 μmol/L; a working concentration of the primer pair targeting rs140202531 is 0.0458 μmol/L; a working concentration of the primer pair targeting rs10573591 is 0.0678 μmol/L; a working concentration of the primer pair targeting rs10630253 is 0.0898 μmol/L; a working concentration of the primer pair targeting rs77624782 is 0.0989 μmol/L; a working concentration of the primer pair targeting rs141471313 is 0.1869 μmol/L; a working concentration of the primer pair targeting rs10600917 is 0.0733 μmol/L; a working concentration of the primer pair targeting rs71408252 is 0.0421 μmol/L; a working concentration of the primer pair targeting rs1404627509 is 0.0293 μmol/L; a working concentration of the primer pair targeting rs74816196 is 0.0421 μmol/L; a working concentration of the primer pair targeting rs112473811 is 0.0825 μmol/L; a working concentration of the primer pair targeting rs57051438 is 0.1154 μmol/L; a working concentration of the primer pair targeting rs766586871 is 0.0679 μmol/L; a working concentration of the primer pair targeting rs10628367 is 0.0679 μmol/L; a working concentration of the primer pair targeting rs58227077 is 0.1274 μmol/L; a working concentration of the primer pair targeting rs143267128 is 0.0764 μmol/L; a working concentration of the primer pair targeting rs140671911 is 0.0849 μmol/L; a working concentration of the primer pair targeting rs138465422 is 0.0340 μmol/L; a working concentration of the primer pair targeting rs200935491 is 0.0679 μmol/L; a working concentration of the primer pair targeting rs141613931 is 0.1076 μmol/L; a working concentration of the primer pair targeting rs66462883 is 0.0736 μmol/L; a working concentration of the primer pair targeting rs71110898 is 0.1104 μmol/L; a working concentration of the primer pair targeting rs35991174 is 0.0651 μmol/L; a working concentration of the primer pair targeting rs35880452 is 0.0708 μmol/L; a working concentration of the primer pair targeting rs59377169 is 0.0758 μmol/L; a working concentration of the primer pair targeting rs778835021 is 0.0884 μmol/L; a working concentration of the primer pair targeting rs79710335 is 0.0455 μmol/L; a working concentration of the primer pair targeting rs59605350 is 0.0758 μmol/L; a working concentration of the primer pair targeting rs112524265 is 0.0379 μmol/L; a working concentration of the primer pair targeting rs5886296 is 0.0303 μmol/L; a working concentration of the primer pair targeting rs59218555 is 0.0425 μmol/L; a working concentration of the primer pair targeting rs67579111 is 0.0474 μmol/L; a working concentration of the primer pair targeting rs71004215 is 0.0278 μmol/L; a working concentration of the primer pair targeting rs56358449 is 0.0360 μmol/L; a working concentration of the primer pair targeting rs140200174 is 0.0343 μmol/L; a working concentration of the primer pair targeting rs56783915 is 0.0425 μmol/L; and a working concentration of the primer pair targeting rs141047228 is 0.0409 μmol/L.

4. The system according to claim 2, wherein, at least one primer of each the primer pair is labelled at 5′ terminal with a fluorescent dye.

5. The system according to claim 4, wherein, the fluorescent dye is selected from the group consisting of FAM, HEX, SUM, LYN, PUR, A514, TAMRA, ROX, VIC, A555, PET, NED, TAZ, A488, SF488 and A568.

6. The system according to claim 4, wherein, the primers with sequences as shown in SEQ ID No:1 to SEQ ID No:30 are set as cluster I, the primers with sequences as shown in SEQ ID No:31 to SEQ ID No:62 are set as cluster II, the primers with sequences as shown in SEQ ID No:63 to SEQ ID No:88 are set as cluster III, the primers with sequences as shown in SEQ ID No:89 to SEQ ID No:118 are set as cluster IV, and the primers with sequences as shown in SEQ ID No:119 to SEQ ID No:146 are set as cluster V; and
each of the clusters is assigned one specific fluorescent dye, and the specific fluorescent dyes assigned to each of the clusters are different from each other.

7. The system according to claim 6, wherein, the fluorescent dye assigned to the cluster I is FAM, the fluorescent dye assigned to the cluster II is HEX, the fluorescent dye assigned to the cluster III is SUM, the fluorescent dye assigned to the cluster IV is LYN, and the fluorescent dye assigned to the cluster V is PUR.

8. The system according to claim 1, wherein, the amplification premix composition comprise dNTPs, Taq DNA Polymerase, Tris-HCl buffer, KCl, MgCl₂ and bovine serum albumin.

9. A kit, comprising the system for multiplex amplification and detection according to claim 1, and a control standard.

10. The kit according to claim 9, wherein, the control standard comprises a positive quality control and/or a negative quality control.

11. The kit according to claim 10, wherein, the positive quality control comprises a mixture of amplification products of the corresponding allelic fragments at each locus.

12. The kit according to claim 10, wherein, the negative quality control comprises nuclease-free water.

13. A method for forensic biogeographical ancestry inference and differentiation of a sample from a subject among the African, European, East Asian, South Asian and South American populations, or Han Chinese, Southeast Asian and Japanese populations, comprising performing nucleic acid amplification on the sample using the system for multiplex amplification and detection according to claim 1, to obtain an amplification product.

14. The method according to claim 10, further comprising subjecting the amplification product to detection.

15. A method for forensic biogeographical ancestry inference and differentiation of a sample from a subject among the African, European, East Asian, South Asian and South American populations, or Han Chinese, Southeast Asian and Japanese populations, comprising performing nucleic acid amplification on the sample using the kit according to claim 10, to obtain an amplification product.

16. The method according to claim 15, further comprising subjecting the amplification product to detection.
